(19) United States
(12) Patent Application Publication
(10) Pub. No.: US 2025/0213765 A1
(43) Pub. Date: Jul. 3, 2025

(54) ELASTOMERIC DOOR FOR MEDICAL FLUID TREATMENT MACHINE

(71) Applicants: BAXTER INTERNATIONAL INC., Deerfield, IL (US); BAXTER HEALTHCARE SA, Glattpark (Opfikon) (CH)

(72) Inventors: Hemanth Hiriyur RAVIKUMAR, Bengaluru (IN); Sunkad SACHIN, Bengaluru (IN); Akhilesh Sham Sundar VIBHUTE, Kalaburagi (IN); Raj Makam Ravindranath APORVA, Shivamogga (IN)

(21) Appl. No.: 18/990,132

(22) Filed: Dec. 20, 2024

(30) Foreign Application Priority Data
Dec. 29, 2023 (IN) 202341089716

(51) Int. Cl. A61M 1/28; A61M 1/14

(52) U.S. Cl. CPC A61M 1/28; A61M 1/156

(57) ABSTRACT

Provided herein is a peritoneal dialysis (“PD”) system comprising a housing and one or more doors comprising elastomeric properties. Each of the one or more doors comprises one or more features configured to couple to one or more features of the housing. Further, each door includes one or more inserts molded between two layers of elastomeric material where the features are coupled to. The doors are configured to cover and thermally insulate one or more PD fluid lines when in a closed configuration. The doors are also configured to transition to an open configuration to provide access to one or more PD fluid lines for treatment.

### PRIORITY CLAIM

This application claims priority to Indian Provisional Application No. 20/2341089716, filed on Dec. 29, 2023, the entire disclosure of which is incorporated herein by reference.

### TECHNICAL FIELD

The present disclosure relates generally to medical fluid treatments, and in particular to housing of medical fluid treatment machines.

### BACKGROUND

Due to various causes, a person's renal system can fail. Renal failure produces several physiological derangements. For instance, it is no longer possible to balance water and minerals or to excrete daily metabolic load. Additionally, toxic end products of metabolism, such as urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for the replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill, and dwell cycles. Automated PD machines, however, perform the cycles automatically, typically while the patient sleeps. The PD machines free patients from having to manually perform the treatment cycles and from having to transport supplies during the day. The PD machines connect fluidly to an implanted catheter, to a source or bag of fresh dialysis fluid and to a fluid drain. The PD machines pump fresh dialysis fluid from a dialysis fluid source, through the catheter and into the patient's peritoneal chamber. The PD machines also allow for the dialysis fluid to dwell within the chamber and for the transfer of waste, toxins, and excess water to take place. The source may include multiple liters of dialysis fluid including several solution bags.

APD machines pump used or spent dialysate from the patient's peritoneal cavity, though the catheter, to drain. As with the manual process, several drain, fill, and dwell cycles occur during dialysis. A “last fill” may occur at the end of an APD treatment. The last fill fluid may remain in the peritoneal chamber of the patient until the start of the next treatment, or may be manually emptied at some point during the day.

Machines for dialysis treatment may include a housing to protect the components used for treatment. The housing may include mechanisms for storing patient lines and additional fluid lines (e.g. fluid lines configured to connect to solution sources) when not in use. The fluid lines may be stored on the housing or may be part of a disposable cassette that is removable from the housing. Storing fluid lines on the housing can be problematic since the fluid lines can be damaged or dislodged through inadvertent contact by a user. Further, it can be difficult to clean the fluid lines and the housing around the fluid lines. Disposable cassettes are generally cumbersome to handle and install in a dialysis machine. A dialysis machine with an improved housing for on board fluid line storage is accordingly needed.

### SUMMARY

Aspects of the subject matter described herein may be useful alone or in combination with one or more other aspects described herein. Without limiting the foregoing description, in a first aspect of the present disclosure, which may be combined with any other aspect, or portion thereof, a peritoneal dialysis (“PD”) system includes a housing with one or more apertures configured to receive one or more fluid lines; and one or more doors including elastomeric properties. Each of the one or more doors includes one or more features configured to couple to one or more features of the housing.

In a second aspect of the present disclosure, which may be combined with any other aspect, or portion thereof, the one or more doors includes one or more magnets configured to secure the one or more doors to the housing.

In a third aspect of the present disclosure, which may be combined with any other aspect, or portion thereof, the housing includes one or more hall sensors configured to detect when the one or more doors are in a closed position.

In a fourth aspect of the present disclosure, which may be combined with any other aspect, or portion thereof, the one or more doors includes two doors.

In a fifth aspect of the present disclosure, which may be combined with any other aspect, the one or more doors comprises at least one insert, wherein the one or more features are coupled to the at least one insert.

In a sixth aspect of the present disclosure, which may be combined with any other aspect, the one or more features are removably coupled to the at least one insert.

In a seventh aspect of the present disclosure, which may be combined with any other aspect, the one or more doors are made of two sheets of elastomeric material, wherein the at least one insert is molded between the two sheets of elastomeric material.

In an eighth aspect of the present disclosure, which may be combined with any other aspect, one insert of the at least one insert includes a ferromagnetic material, wherein the one or more doors include one or more magnets, and wherein the one or more magnets are configured to couple to the one insert to hold the one or more doors in an open configuration.

In a ninth aspect of the present disclosure, which may be combined with any other aspect, the one or more features of the door includes one or more rivets, and the one or more features of the housing includes one or more counter holes configured to receive the one or more rivets.

In a tenth aspect of the present disclosure, which may be combined with any other aspect, in a closed configuration, the one or more doors are configured to cover the one or more apertures configured to receive the one or more fluid lines.

In an eleventh aspect of the present disclosure, which may be combined with any other aspect, a peritoneal dialysis (“PD”) system includes a housing with one or more apertures configured to receive one or more fluid lines; and one or more doors with elastomeric properties, wherein the one or more doors are coupled to the housing using one or more magnets.

In a twelfth aspect of the present disclosure, which may be combined with any other aspect, the one or more doors include the one or more magnets and the housing includes a ferromagnetic material configured to couple to the one or more magnets to secure the one or more doors to the housing.

In a thirteenth aspect of the present disclosure, which may be combined with any other aspect, the housing includes one or more magnets and the one or more doors includes a ferromagnetic insert configured to couple the one or more doors to the housing.

In a fourteenth aspect of the present disclosure, which may be combined with any other aspect, the housing and the one or more doors include the one or more magnets, and the one or more magnets of the one or more doors are configured to couple to the one or more magnets of the housing.

In a fifteenth aspect of the present disclosure, which may be combined with any other aspect, the one or more doors include at least two layers of elastomeric material, wherein the one or more magnets are molded between the at least two layers elastomeric material.

In a sixteenth aspect of the present disclosure, which may be combined with any other aspect, wherein the one or more magnets include a first set of magnets with a first magnetic force and a second set of magnets with a second magnetic force.

In a seventeenth aspect of the present disclosure, which may be combined with any other aspect, the first set of magnets include a higher magnetic retention force than the second set of magnets.

In an eighteenth aspect of the present disclosure, which may be combined with any other aspect, in a closed configuration, the one or more doors are configured to cover the one or more apertures configured to receive the one or more fluid lines.

In a nineteenth aspect of the present disclosure, which may be combined with any other aspect, the housing includes one or more hall sensors configured to detect when the one or more doors are in a closed configuration.

In a twentieth aspect of the present disclosure, which may be combined with any other aspect, the PD system further includes a user interface, wherein the one or more doors surround the user interface in a closed configuration.

In light of the above aspects and present disclosure set forth herein, it is an advantage of the present disclosure to provide a PD system with doors that provide thermal insulation of disinfectant fluid in PD fluid lines.

It is another advantage of the present disclosure to provide a PD system that has doors that are removable from housing without the use of any tool.

It is a further advantage of the present disclosure to provide a PD system that has removable parts for coupling the door to the housing that can be replaced upon damage without having to replace the whole door.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a medical fluid, e.g., PD fluid, system that is set for treatment.

FIG. 2 illustrates one embodiment of a PD system with doors in a closed configuration according to the present disclosure.

FIG. 3 illustrates one embodiment of a PD system with doors in an open configuration according to the present disclosure.

FIG. 4 illustrates one embodiment of a PD system set for treatment with doors in an open configuration according to the present disclosure.

FIG. 5 illustrates one embodiment of a door according to an example of the present disclosure.

FIG. 6 illustrates an exploded view of PD system of housing with doors according to the present disclosure.

FIG. 7 illustrates a top perspective view of the PD system of FIG. 6.

FIG. 8 illustrates a cross-sectional view of the PD system of FIG. 6 with a rivet interfacing a counter hole of housing according to the present disclosure.

FIG. 9 illustrates an insert coupled to housing according to an example of the present disclosure.

FIG. 10 illustrates an internal view of housing according to an example of the present disclosure.

FIG. 11 illustrates a PD system showing an embodiment of doors affixing to housing according to the present disclosure.

FIG. 12 illustrates a PD system showing an embodiment of doors affixed to housing according to the present disclosure.

### DETAILED DESCRIPTION

### System Overview

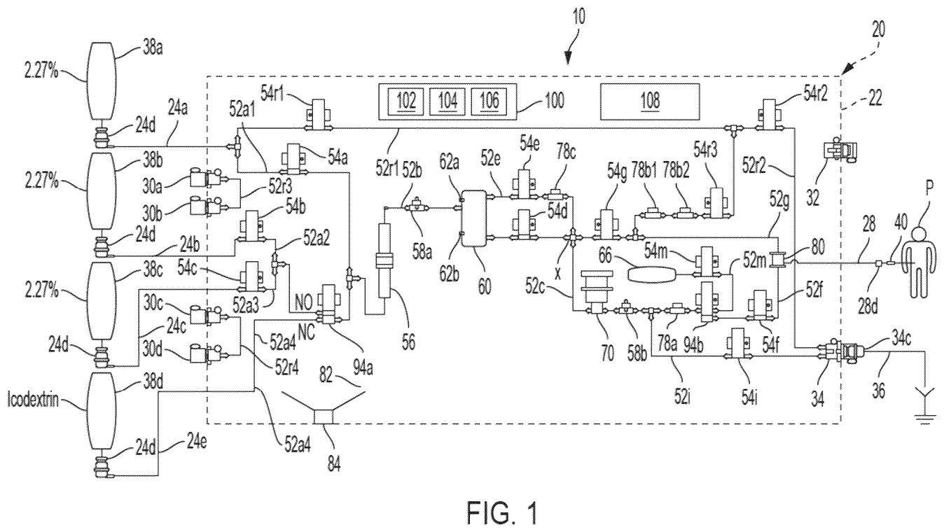

Referring now to the drawings and in particular to FIG. 1, a peritoneal dialysis (“PD”) system 10 according to the present disclosure is illustrated. System 10 includes a PD machine or cycler 20 and a control unit 100 having one or more processor 102, one or more memory 104, video controller 106, and user interface 108. The example user interface 108 may alternatively or additionally be a remote user interface, e.g., via a tablet or smartphone. The control unit 100 may also include a transceiver and a wired or wireless connection to a network (not illustrated), e.g., the internet, for sending treatment data to and receiving prescription instructions/changes from a doctor's or clinician's server interfacing with a doctor's or clinician's computer. The control unit 100 in an embodiment controls all electrical, fluid flow, and heating components of the system 10 and receives outputs from sensors of the system 10. System 10 in the illustrated embodiment includes durable and reusable components that contact fresh and used PD fluid, which necessitates that the PD machine or cycler 20 be disinfected between treatments, e.g., via heat disinfection.

System 10 in FIG. 1 includes an inline resistive heater 56, reusable supply lines or tubes 52a1 to 52a4 and 52b, air trap 60 operating with respective upper and lower level sensors 62a and 62b, air trap valve 54d, vent valve 54e located along vent line 52e, reusable line or tubing 52c, PD fluid pump 70, temperature sensors 58a and 58b, pressure sensors 78a, 78b1, 78b2 and 78c, reusable patient tubing or lines 52f and 52g having respective valves 54f and 54g, dual lumen reusable patient line 28, a hose reel 80 for retracting patient line 28, reusable drain tubing or line 52i extending to drain line connector 34 and having a drain line valve 54i, and reusable recirculation disinfection tubing or lines 52rl and 52r2 operating with respective disinfection valves 54rl and 54r2. A third recirculation or disinfection tubing or line 52r3 extends between disinfection connectors 30a and 30b for use during disinfection. A fourth recirculation or disinfection tubing or line 52r4 extends between disinfection connectors 30c and 30d for use during disinfection.

System 10 further includes PD fluid containers or bags 38a to 38c (e.g., holding the same or different formulations of PD fluid), which connect to distal ends 24d of reusable PD fluid lines 24a to 24c, respectively. System 10 further includes a fourth PD fluid container or bag 38d that connects to a distal end 24d of reusable PD fluid line 24e. Fourth PD fluid container or bag 38d may hold the same or different type (e.g., icodextrin) of PD fluid than provided in PD fluid containers or bags 38a to 38c. Reusable PD fluid lines 24a to 24c and 24e extend in one embodiment through apertures defined or provided by the housing 22 of the cycler 20.

System 10 in the illustrated embodiment includes four disinfection connectors 30a to 30d for connecting to distal ends 24d of reusable PD fluid lines 24a to 24c and 24e, respectively, during disinfection. System 10 also provides a patient line connector 32 that includes an internal lumen, e.g., a U-shaped lumen, which for disinfection directs fresh or used dialysis fluid from one PD fluid lumen of a connected distal end 28d of dual lumen reusable patient line 28 into the other PD fluid lumen. Reusable supply tubing or lines 52al to 52a4 communicate with reusable supply lines 24a to 24c and 24e, respectively. Reusable supply tubing or lines 52a1 to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

System 10 is constructed in one embodiment such that drain line 52i during a patient fill is fluidly connected downstream from PD fluid pump 70. In this manner, if drain valve 54i fails or somehow leaks during the patient fill of patient P, fresh PD fluid is pushed down disposable drain line 36 instead of used PD fluid potentially being pulled into pump 70. Disposable drain line 36 is in one embodiment removed for disinfection, wherein drain line connector 34 is capped via a cap 34c to form a closed disinfection loop. PD fluid pump 70 may be an inherently accurate pump, such as a piston pump, or less accurate pump, such as a gear pump that operates in cooperation with a flowmeter (not illustrated) to control fresh and used PD fluid flowrate and volume.

System 10 may further include a leak detection pan 82 located at the bottom of housing 22 of cycler 20 and a corresponding leak detection sensor 84 outputting to control unit 100. In the illustrated example, system 10 is provided with an additional pressure sensor 78c located upstream of PD fluid pump 70, which allows for the measurement of the suction pressure of pump 70 to help control unit 100 more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control while the output of the other pressure sensor is a safety or watchdog output to make sure the control pressure sensor is reading accurately. Pressure sensors 78b1 and 78b2 are located along a line including a third recirculation valve 54r3.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between citric acid container or bag 66 and the PD fluid circuit during treatment. The redundant valves ensure that no citric (or other) acid reaches the treatment fluid lines during treatment. Citric (or other) acid is instead used during disinfection.

Control unit 100 in an embodiment uses feedback from any one or more of pressure sensors 78a to 78c to enable PD machine 20 to deliver fresh, heated PD fluid to the patient at, for example, 14 kPa (2.0 psig) or higher. The pressure feedback is used to enable PD machine 20 to remove used PD fluid or effluent from the patient at, for example, −9 kPa (−1.3 psig) or higher. The pressure feedback may be used in a proportional, integral, derivative (“PID”) pressure routine for pumping fresh and used PD fluid at a desired positive or negative pressure.

Inline resistive heater 56 under control of control unit 100 is capable of heating fresh PD fluid to body temperature, e.g., 37° C., for delivery to patient P at a desired flowrate. Control unit 100 in an embodiment uses feedback from temperature sensor 58a in a PID temperature routine for pumping fresh PD fluid to patient P at a desired temperature.

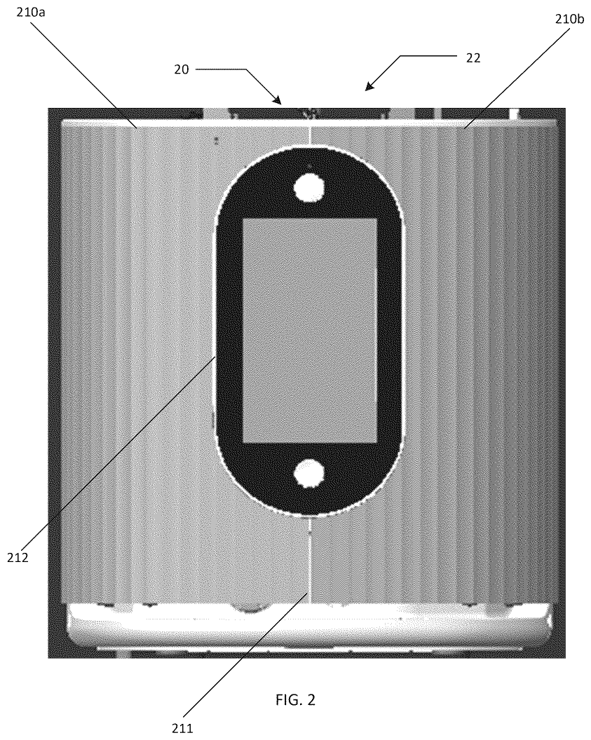

FIG. 1 illustrates system 10 setup for treatment with PD fluid containers or bags 38a to 38d connected via reusable, flexible PD fluid lines 24a to 24d, respectively. Dual lumen patient line 28 is connected to patient P via disposable filter set 40. Disposable drain line 36 is connected to drain line connector 34. In FIGS. 1 and 2, PD machine or cycler 20 of system 10 is configured to perform multiple patient drains, patient fills, patient dwells, and a priming procedure, as part of or in preparation for treatment.

For example, during patient fills, the PD machine or cycler 20 is configured to pump fresh dialysis fluid from PD fluid containers or bags 38a to 38d, through the patient line 28, through the disposable filter set, and into the patient's peritoneal chamber. The PD machines also allow for the dialysis fluid to dwell within the chamber and for the transfer of waste, toxins, and excess water to take place. The source may include multiple liters of dialysis fluid including several solution bags. During patient drain, the PD machine or cycler 20 pumps used or spent dialysate from the patient's peritoneal cavity, though the disposable transfer set, through the patient line, and to the drain.

### Housing with Elastomeric Doors

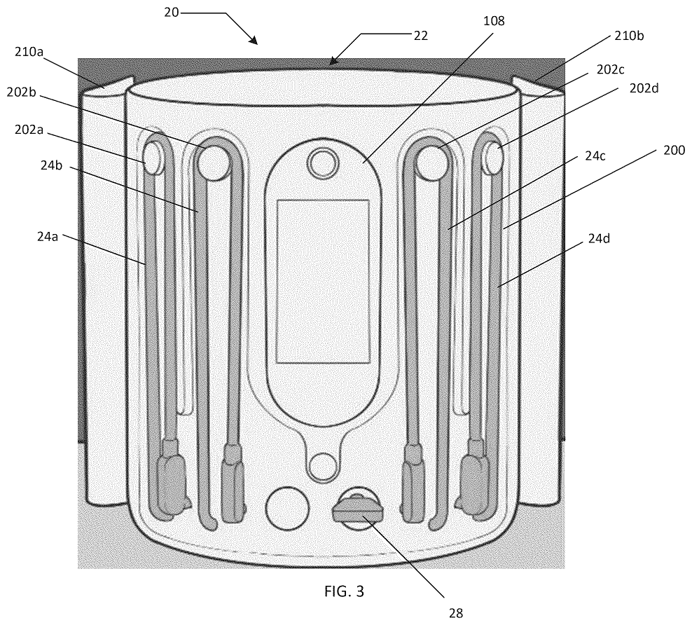

FIGS. 2 and 3 illustrate housing 22 of cycler 20 according to an example of the present disclosure. System 10 as described above may be contained wholly or partially within the housing 22. For example, in some embodiments, the control system 100, the inline resistive heater 56, reusable supply lines or tubes 52al to 52a4 and 52b, the air trap 60, the air trap valve 54d, the vent valve 54e, the vent line 52e, the reusable line or tubing 52c, the PD fluid pump 70, temperature sensors 58a and 58b, pressure sensors 78a, 78b1, 78b2 and 78c, reusable patient tubing or lines 52f and 52g and respective valves 54f and 54g, dual lumen reusable patient line 28, the hose reel 80 for retracting patient line 28, reusable drain tubing or line 52i extending to drain line connector 34, a drain line valve 54i, reusable recirculation disinfection tubing or lines 52r1 and 52r2, and/or respective disinfection valves 54r1 and 54r2 are contained within housing 22. In one embodiment, housing 22 of cycler 20 is made of plastic and/or of metal, e.g., stainless steel, steel and/or aluminum, to protect internal components of cycler 20.

As illustrated in FIG. 2, housing 22 of cycler 20 is provided with one or more doors or jackets 210a, 210b that are configured to partially or fully surround the outside of housing 22. In some embodiments, the outside surface of door 210a, 210b includes ribs. The ribs may provide structural support to the doors 210a. Alternatively, the outside surface of the door 210a includes a smooth surface or other type of textured surface. When doors 210a, 210b are in a closed configuration as shown in FIG. 2, doors 210a, 210b cover the patient line 28 and the PD fluid lines 24a to 24d.

During the disinfection cycle, hot solution flows through the fluid lines, which may be accidentally touched by a patient or clinician. Doors 210a, 210b are configured to provide thermal insulation to heated PD fluid lines 24a to 24d during disinfection. The doors 210a, 210b also prevent a patient or user from contacting heated medical fluid during disinfection, which could cause harm.

In the depicted embodiment, at least a portion of one edge 211 of each door 210a, 210b meet together when the doors 210a, 210b are in a closed configuration. The other portion of one edge 212 of each door 210a, 210b surrounds the user interface 108 so the user can access the user interface 108 while the doors 210a, 210b are closed. However, it can be appreciated that in some embodiments, the cycler 20 includes one door surrounding housing 22 or two or more doors 210a, 210b that meet together along one full edge. In other embodiments, the doors 201a, 210b may have any other suitable configuration.

FIG. 3 illustrates the doors 210a, 210b in an open configuration. To prepare for treatment, a user opens or removes doors 210a, 210b to access the patient line 28 and PD fluid lines 24a to 24d. As illustrated in FIG. 3, reusable PD fluid lines 24a to 24d and/or patient line 28 extend from inside the housing 22 through one or more apertures to the outside of the housing to be accessed by the user. Housing 22 may be provided with a recessed surface 200 for storing PD fluid lines 24a to 24d and patient line 28. The recessed surface 200 allows fluid lines to be stored while also allowing doors to lay flat on housing 22 when closed.

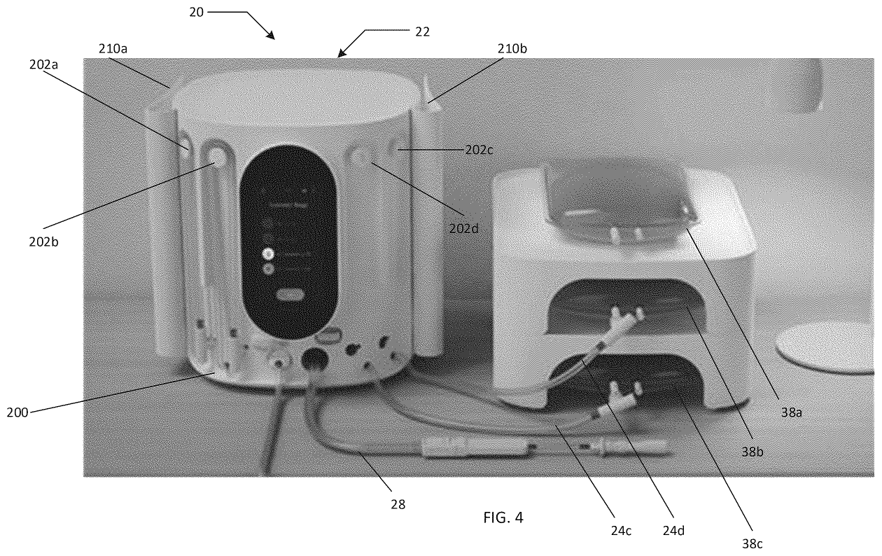

Additionally or alternatively, housing 22 provides one or more holders 202a to 202d to hold the PD fluid lines 24a to 24d during storage or disinfection. Before treatment, a user removes one or more PD fluid lines 24a to 24d from holders 202a to 202d to connect the one or more PD fluid lines 24a to 24d to PD fluid containers or bags 38a to 38c as illustrated in FIG. 4. Further, the user unwinds patient line 28 from reel to connect patient line 28 to patient. A user can replace the PD fluid lines 24a to 24d on the respective holder 202a to 202d and retract patient line 28 after use by patient, such as for disinfection.

The doors 210a, 210b are configured to be moved from an open configuration to a closed configuration depending on the desired operation of cycler 20. As illustrated in FIGS. 3 and 4, in the open configuration, the doors are configured to bend backward to expose the patient line 28 and/or the PD fluid lines 24a to 24d. The doors 210a, 210b of cycler 20 have elastomeric properties allowing the doors to bend under stress applied by the user. Further, the elastomeric properties allow the doors to return to an original, complimentary shape of housing 22 when at rest in a closed configuration. In some embodiments, the one or more doors are made out of one or more elastomeric materials, such a silicone or rubber, which impart the elastomeric properties to the doors 210a, 210b.

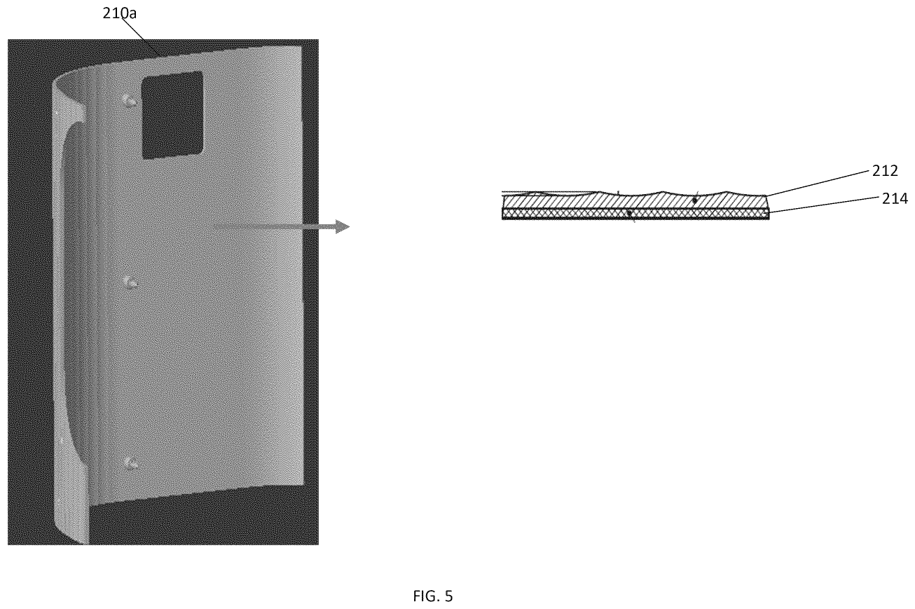

FIG. 5 illustrates an embodiment of a door 210a according to an example of the present disclosure. In some embodiments, the doors 210a, 210b are manufactured by bonding two sheets 212, 214 of elastomeric material together. It can be appreciated that in other embodiments, the doors are manufactured using one, two, three, or more sheets of elastomeric material. In some embodiments, the elastomeric sheets 212, 214 are composed of one or more of liquid silicone rubber, solid silicone rubber, thermoplastic polyurethane, industrial textile fabric, etc. In some instances, a combination of liquid silicone rubber and solid silicone rubber may be used since it is robust (e.g., inert) against disinfectants and cleaning solutions. In some embodiments, the elastomeric material is vulcanized.

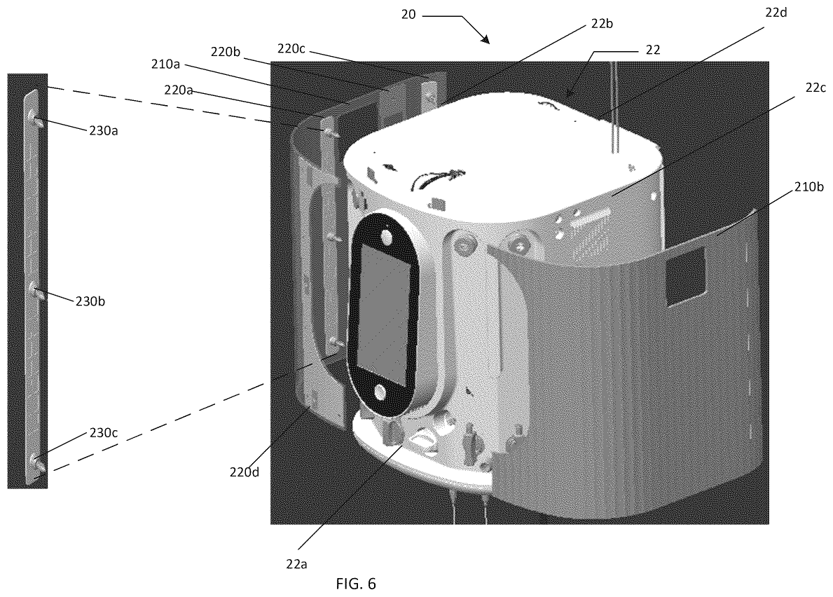

FIGS. 6 to 8 illustrate an interface of doors 210a, 210b with housing 22 according to an example of the present disclosure. As illustrated in FIG. 6, housing 22 includes a front surface 22a, two side surfaces 22b, 22c, and a back surface 22d. The front surface 22a includes the user interface 108 for controlling cycler 20. The front surface 22a further includes the recessed surface 200 and holders 202a to 202d for storing PD fluid lines. In the closed configuration, doors 210a, 210b cover at least the front surface 22a and the side surfaces 22b, 22c of housing 22. In the open configuration, the portion of doors 210a, 210b covering front surface 22a and PD fluid lines are bent away from housing 22 so doors 210a, 210b only cover side surfaces 22b, 22c. In other embodiments, doors are fully removed to allow access to PD fluid lines for therapy.

In some embodiments, the doors 210a, 210b affix to housing 22 on the side surfaces of housing 22. In some embodiments, the doors 210a, 210b affix to the housing 22 on the front surface 22a, the side surfaces 22b, 22c, and/or the back surface 22d. Preferably, doors 210a, 210b connect to housing 22 without the use of hinges, which are more likely to wear after continued use. In some embodiments, the doors 210a, 210b affix to housing 22 using one or more inserts 220a to 220d. The inserts 220a to 220d may be composed of a rigid material, such as plastic or metal, to impart structure to the elastomeric doors. In the depicted embodiment, the inserts 220a to 220d are molded into one or more layers of the elastomeric material of door 210a, 210b. Molding the inserts 220a to 220d into the door 210a, 210b leads to better strength and holding force when compared to bonding the inserts 220a to 220d to the surface of the door 210a, 210b. In alternative embodiments, the one or more inserts 220a to 220d are bonded to an outside surface of the door 210a, 210b. One or all inserts 220a to 220d include one or more features configured to mate with one or more features of housing 22.

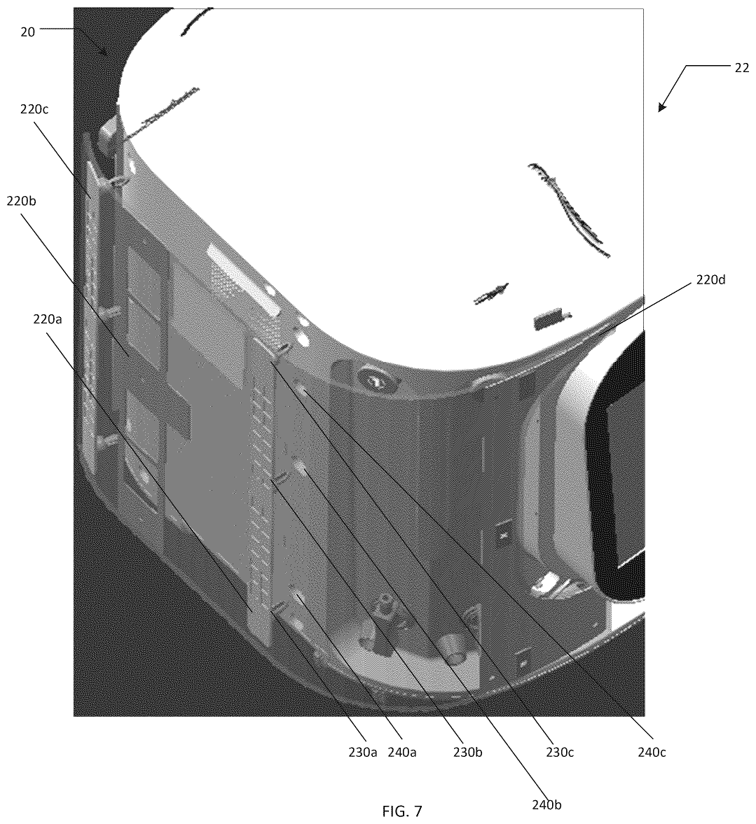

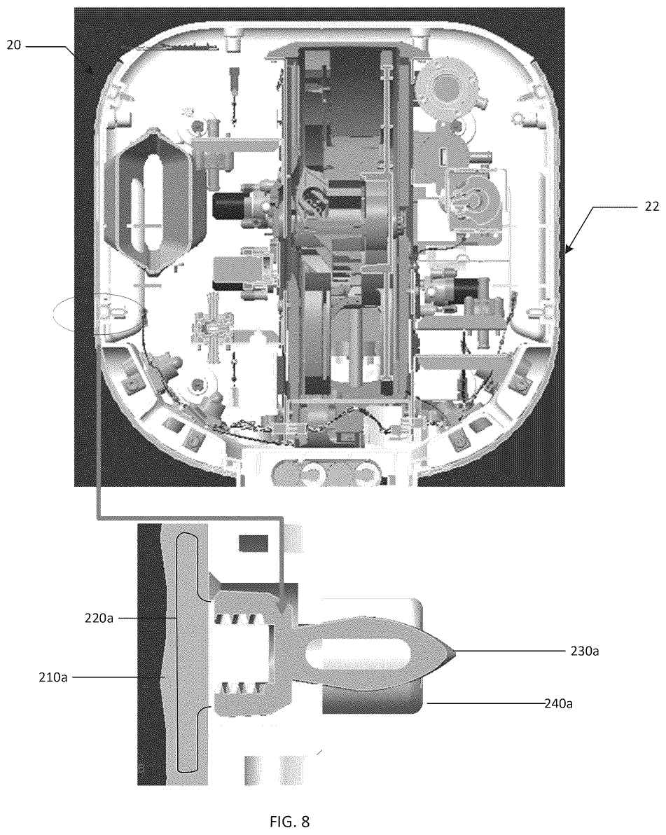

Referring to FIG. 6, in some embodiments, the one or more features include one or more rivets 230a to 230c directly or indirectly coupled to the insert 220a to 220d. The one or more rivets may extend from an internal surface of the door 210a, 210b. As shown in FIGS. 7 and 8, housing 22 includes one or more counter holes 240a to 240c configured to receive the one or more rivets 230a to 230c. In some embodiments, the rivets 230a to 230c are directly or indirectly coupled to housing 22, while the door 210a, 210b includes the one or more counter holes 240a to 240c. While rivets 230a to 230c and counter holes 240a to 240c are shown, it can be appreciated that other features can be used to affix the doors 210a, 210b to housing 22.

As depicted in FIG. 8, the rivet 230a may be removably coupled to the insert 220a, such as through the use of a threaded engagement. Alternatively, other removable coupling arrangements are used. In the event that one of the rivets 230a to 230c becomes damaged, a user can easily remove and replace the rivet 230a to 230c without the need to replace the entire door 210a, 210b. However, it can be appreciated that the rivets 230a to 230c may be formed integrally with the door 210a, 210b and/or insert 220a to 220d.

As shown in FIGS. 6 and 7, each door 210a, 210b may include a plurality of inserts 220a to 220d for securing doors 210a, 210b to housing. In some embodiments, the one or more inserts 220a to 220d do not include any securement features, but are instead configured for structural stability. In some embodiments, each insert 220a to 220d includes different or same securement features as other inserts 220a to 220d. For example, insert 220a and insert 220c both include rivets for connecting to counter holes 240a to 240c in side surfaces of housing 22 as described previously. Contrastingly, insert 220d secures to front surface of housing 22, such as around user interface 108, using one or more magnets. Magnets allow easier opening and closing of doors 210a, 210b during treatment and disinfection while rivets 230a to 230c provide a more stable securement. However, the entire door 210a, 210b can still be removed manually without the use of a tool. It can be appreciated that other combinations of securement features may be used.

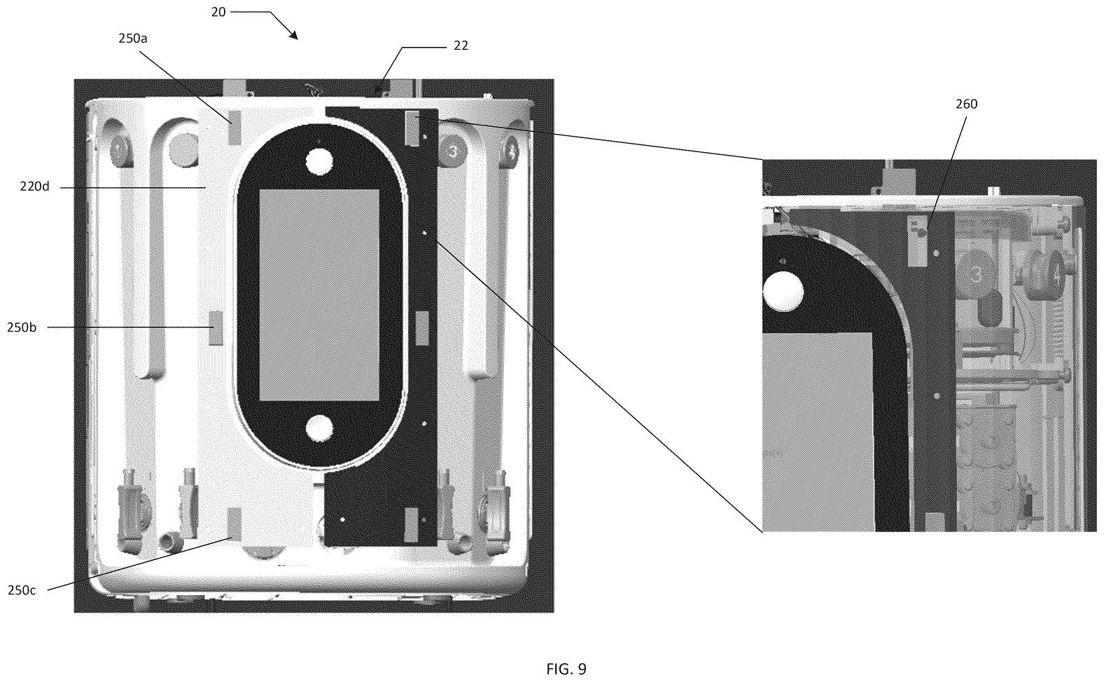

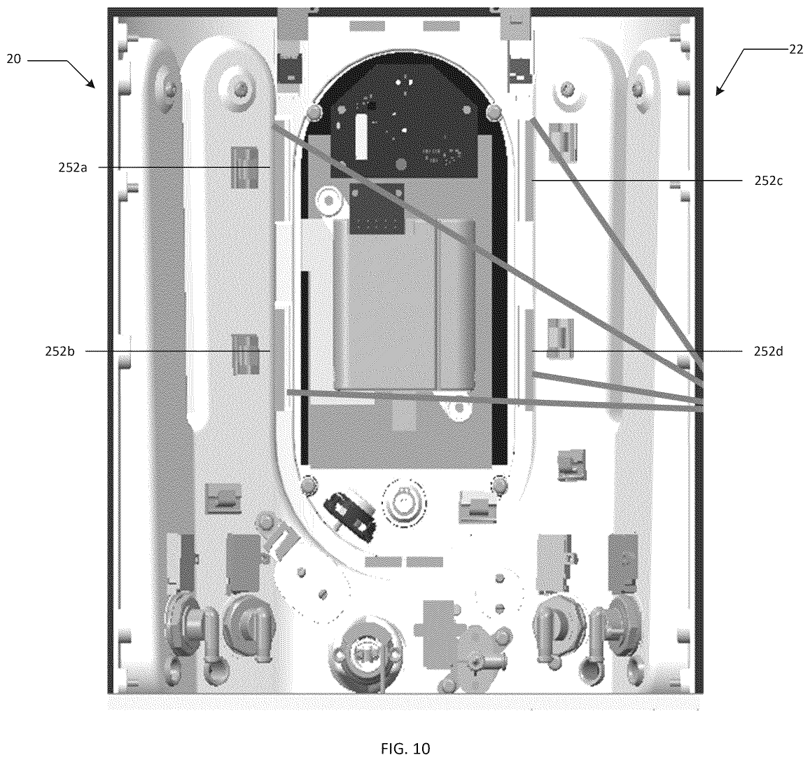

FIG. 9 illustrates insert 220d coupled to housing 22 with doors 210a, 210b not shown. The one or more magnets 250a to 250c may be coupled to insert 220d or molded into doors 210a, 210b. Housing 22 includes one or more ferromagnetic materials, such as iron, cobalt, steel, nickel, and the like, for coupling the insert 220d to housing 22. Alternatively, as shown in FIG. 10, housing 22 includes one or more magnets (e.g. on inside surface of housing 22) and doors 210a, 210b include an insert made of a ferromagnetic material. The magnets adhere to the ferromagnetic component of the door 210a, 210b through a magnetic retention force. In some embodiments, both the housing 22 and doors 210a, 210b include one or magnets configured to attract each other to secure the door on the housing.

Housing 22 further includes a sensor, such as a hall sensor 260, for detecting if the door 210a, 210b is in an open or closed configuration as shown in FIG. 9. In some embodiments, the user interface provides the user a notification if the doors 210a, 210b are not properly closed before disinfection. Additionally or alternatively, in some embodiments, the user cannot enter the disinfection cycle until the hall sensor 260 detects the door 210a, 210b in the closed position.

Referring back to FIGS. 6 and 7, doors 210a, 210b may further include insert 220b including one or more magnets and/or a ferromagnetic material. As discussed previously, insert 220d may also include a magnet and/or a ferromagnetic material. A portion of insert 220d may be configured to couple to insert 220b through magnetic retention force to hold doors 210a, 210b in an open position (as illustrated in FIG. 3). A user can easily couple and uncouple doors 210a, 210b from an open and closed position depending on operation of cycler.

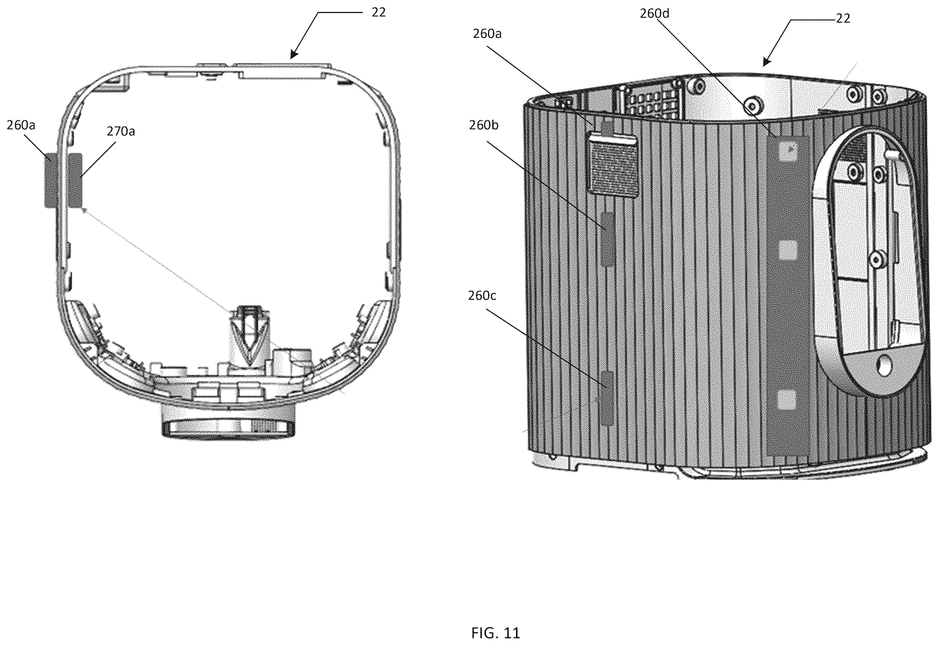

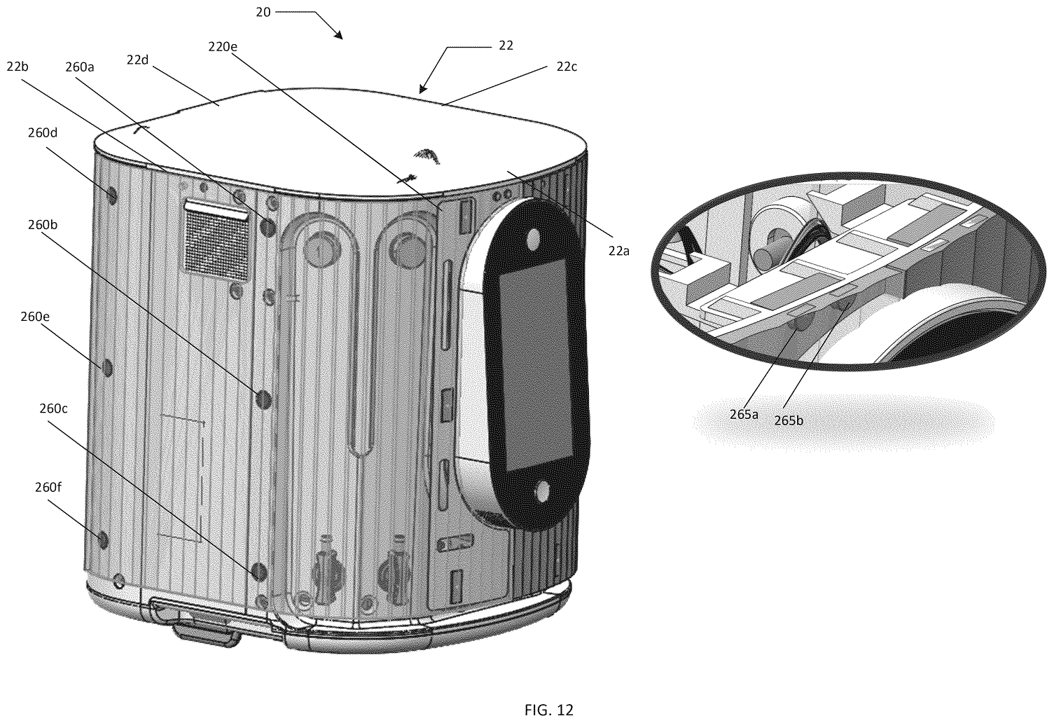

FIGS. 11 and 12 illustrate an interface of doors 210a, 210b with housing 22 according to another example of the present disclosure. In the depicted embodiment, doors 210a, 210b are affixed to housing using one or more magnets. Doors 210a, 210b include one or more magnets 260a to 260d configured to attract to one or more magnets 270a of housing 22. In some embodiments, the magnets 260a to 260c are molded into the elastomeric material of doors 201a, 210b (e.g. between layers of the door). As shown in FIG. 12, in some embodiments, magnets are coupled to one or more inserts 220e of doors 210a, 210b. Inserts 220e also provide additional structural stability to the elastomeric doors 210a, 210b. Alternatively, the magnets 260a to 260c are affixed to a surface of door 210a, 210b. In some embodiments, one of housing 22 or doors 210a, 210b include one or more magnets while one of housing 22 or doors 210a, 210b include a ferromagnetic material (e.g., on insert 220e or panel).

In some embodiments, doors 210a, 210b include magnets configured to secure door to side surfaces 22b, 22c of housing 22 and magnets configured to secure doors 210a, 210b to a front surface 22a of housing 22. As illustrated in FIG. 12, the one or more magnets include a first set of magnets 260a to 260f with one magnetic retention force and a second set of magnets 265a,265b with a different magnetic retention force. A first set of magnets 260a to 260f has a higher retention force (e.g., about 15 N or higher) used to secure door to a side surface 22b of housing 22, while a second set of magnets 265a,265b has a lower retention force (about 1 N to about 7 N, more preferably about 5 N) used to secure door 210a, 210b to a front surface 22a of housing 22.

The use of a lower retention force for securing doors 210a, 210b to front surface 22a allows easier transition of doors 210a, 210b from an open configuration to a closed configuration to access PD fluid lines, while keeping doors 210a, 210b secure on the side surface 22b, 22c using the higher force magnets. Further, magnets are configured to allow a user to easily remove doors 21a, 210b from housing 22. Upon removal of doors 210a, 210b, a user can clean housing 22 while still being able to replace doors 210a, 210b for treatment and/or disinfection.

It should be understood that other various changes and modifications to the presently preferred embodiments described herein will be apparent to those skilled in the art. It is therefore intended that such changes and modifications be covered by the appended claims.

The invention is claimed as follows:

1. A peritoneal dialysis (“PD”) system comprising:
a housing, the housing comprising one or more apertures configured to receive one or more fluid lines; and
one or more doors comprising elastomeric properties, wherein each of the one or more doors comprises one or more features configured to couple to one or more features of the housing.

2. The PD system of claim 1, wherein the one or more doors comprises one or more magnets configured to secure the one or more doors to the housing.

3. The PD system of claim 1, wherein the housing comprises one or more hall sensors configured to detect when the one or more doors are in a closed position.

4. The PD system of claim 3, wherein the one or more doors comprises two doors.

5. The PD system of claim 1, wherein the one or more doors comprises at least one insert, wherein the one or more features are coupled to the at least one insert.

6. The PD system of claim 5, wherein the one or more features are removably coupled to the at least one insert.

7. The PD system of claim 5, wherein the one or more doors comprise two sheets of elastomeric material, wherein the at least one insert is molded between the two sheets of elastomeric material.

8. The PD system of claim 5, wherein one insert of the at least one insert comprises a ferromagnetic material, wherein the one or more doors comprises one or more magnets, and wherein the one or more magnets are configured to couple to the one insert to hold the one or more doors in an open configuration.

9. The PD system of claim 1, wherein the one or more features of the door comprises one or more rivets, and wherein the one or more features of the housing comprises one or more counter holes configured to receive the one or more rivets.

10. The PD system of claim 1, wherein in a closed configuration, the one or more doors are configured to cover the one or more apertures configured to receive the one or more fluid lines.

11. A peritoneal dialysis (“PD”) system comprising:
a housing, the housing comprising one or more apertures configured to receive one or more fluid lines; and
one or more doors comprising elastomeric properties, wherein the one or more doors are coupled to the housing using one or more magnets.

12. The PD system of claim 11, wherein the one or more doors comprise the one or more magnets and wherein the housing comprises a ferromagnetic material configured to couple to the one or more magnets to secure the one or more doors to the housing.

13. The PD system of claim 11, wherein the housing comprises the one or more magnets and the one or more doors comprise a ferromagnetic insert configured to couple the one or more doors to the housing.

14. The PD system of claim 11, wherein the housing and the one or more doors comprise the one or more magnets, and wherein the one or more magnets of the one or more doors are configured to couple to the one or more magnets of the housing.

15. The PD system of claim 11, wherein the one or more doors comprise at least two layers of elastomeric material, wherein the one or more magnets are molded between the at least two layers elastomeric material.

16. The PD system of claim 11, wherein the one or more magnets comprise a first set of magnets with a first magnetic force and a second set of magnets with a second magnetic force.

17. The PD system of claim 16, wherein the first set of magnets comprises a higher magnetic retention force than the second set of magnets.

18. The PD system of claim 11, wherein in a closed configuration, the one or more doors are configured to cover the one or more apertures configured to receive the one or more fluid lines.

19. The PD system of claim 11, wherein the housing comprises one or more hall sensors configured to detect when the one or more doors are in a closed configuration.

20. The PD system of claim 11 further comprising a user interface, wherein the one or more doors surround the user interface in a closed configuration.
